(19) United States
(12) Patent Application Publication
(10) Pub. No.: US 2021/0033610 A1
(43) Pub. Date: Feb. 4, 2021

(54) INTRACELLULAR NITRIC OXIDE AS A BIOMARKER FOR INCREASED SUSCEPTIBILITY SYSTEMIC INFLAMMATION

(71) Applicant: The Research Foundation for the State University of New York, Albany, NY (US)

(72) Inventors: Rauno Joks, Port Washington, NY (US); Maya Nowakowski, Port Washington, NY (US); Helen G. Durkin, Manhasset, NY (US)

(21) Appl. No.: 16/969,093

(22) PCT Filed: Feb. 8, 2019
(86) PCT No.: PCT/US19/17213

§ 371 Date: Aug. 11, 2020

Related U.S. Application Data
(60) Provisional application No. 62/629,576, filed on Feb. 12, 2018.

(51) Int. Cl. G01N 33/573; C12N 5/078

(52) U.S. Cl. CPC G01N 33/573; C12N 5/0634; G01N 2333/70596; G01N 2333/90254; C12N 2501/2315; C12N 2501/2318; C12N 2501/24; C12N 2500/38

(57) ABSTRACT

Methods and kits are provided for identifying subjects at increased risk for IgE mediated systemic inflammatory disorders, particularly asthma.

### CROSS-REFERENCE TO RELATED APPLICATIONS

The present application claims benefit of U.S. Provisional Application No. 62/629,576, filed Feb. 12, 2018, the entire disclosure being incorporated herein by reference.

### FIELD OF THE INVENTION

This invention relates to the fields of biomarkers, inflammatory conditions and disease management. More specifically, methods and kits are provided for identifying subjects at increased risk for systemic inflammatory disease and treating subjects so identified with the appropriate pharmaceutical agent(s).

### BACKGROUND OF THE INVENTION

Several publications and patent documents are cited throughout the specification to describe the state of the art to which this invention pertains. Each of these citations is incorporated herein by reference as though set forth in full.

Nitric oxide (NO) was initially described as a physiological mediator of endothelial cell relaxation, playing an important role in hypotension. NO is also an intercellular messenger that has been recognized as one of the most versatile players in the immune system. Cells of the innate immune system—macrophages, neutrophils and natural killer cells—use pattern recognition receptors to recognize the molecular patterns associated with pathogens. Activated macrophages then inhibit pathogen replication by releasing a variety of effector molecules, including NO. In addition to macrophages, a large number of other immune-system cells produce and respond to NO. Thus, NO is important as a toxic defense molecule against infectious organisms. It also regulates the functional activity, growth and death of many immune and inflammatory cell types including macrophages, T lymphocytes, antigen-presenting cells, mast cells, neutrophils and natural killer cells. Indeed, exhaled NO is often used in methods for the diagnosis of asthma.

As described in Dixon et al. (Am J Respir Crit Care Med (2015) 191:721-722), there is a strikingly high prevalence of obesity among individuals with asthma. Although about one-third of the U.S. population is obese, many recent studies of asthmatic populations report a prevalence of obesity of 50% or more in these individuals. Studies investigating the pathogenesis of asthma in obesity using animal models suggest that obesity causes an asthma-like phenotype through innate, non-Th2 pathways. Indeed, there appears to be a phenotype of asthma, more common in women, with late-onset disease and low levels of markers of allergic inflammation. However, obesity is a risk factor for both atopic and nonatopic asthma. Many obese individuals have elevated serum eosinophils and high IgE levels, and although obesity is typically associated with low sputum eosinophil and low exhaled nitric oxide levels, there may be an abnormality of eosinophil trafficking in obesity, with elevated submucosal (but not sputum) eosinophil levels.

It is clear that NO mediates multiple metabolic processes both in health and disease, yet its measurement has remained problematic. For example, while use of exhaled nitric oxide is an established biomarker of allergic asthma during diagnosis, exhaled NO levels do not correlate with current symptoms, and thus, such measurements, while useful in diagnosis, provides little guidance to the clinician for management of this disease.

### SUMMARY OF THE INVENTION

There have been no clinical assays available to measure blood nitric oxide responses. As nitric oxide is a core immune and vascular mediator in a variety of common diseases, including, without limitation, asthma, hypertension, coronary artery disease, and stroke, the present findings of an association of obesity, a chronic inflammatory condition associated with the above, with peripheral blood nitric oxide responses has significant merit and provides a marker of innate immune response activation contributing to the development and progression of these conditions.

In one embodiment of the invention, a method for detecting iNOS expression levels in CD33⁺ monocytes as a biomarker for uncontrolled asthma in IgE⁺ subjects is provided. An exemplary method comprises obtaining a peripheral blood cell (PBMC) sample from the subject, contacting the PBMC sample with antibodies that are immunospecific for CD33 and intracellular nitric oxide synthase (iNOS), thereby forming detectable immunocomplexes, and measuring the percentage of PBMC in the sample which are both CD33⁺ and iNOS⁺. In preferred embodiments, the CD33⁺ and iNOS⁺ cells are detected using flow cytometry. The present inventors have discovered that the presence of greater than 5%, greater than 10%, greater than 15%, or greater than 20% CD33⁺, iNOS⁺ monocytes in the PBMC preparation is correlated with poor control of asthmatic symptoms in IgE⁺ patients, indicating that these patients are in need of additional therapy to manage these symptoms. See FIG. 4. The method optionally includes determining whether the monocytes express one or more of CD45 and CD14. In a particularly preferred embodiment, the measurement of peripheral blood CD33⁺iNOS⁺ cell levels is employed in a test and treat method to adjust asthma and/or rhino-conjunctivitis therapies.

In another embodiment of the invention, a method for detecting iNOS expression levels in CD33⁺ monocytes as a biomarker for systemic inflammation in IgE⁺ subjects is provided. An exemplary method comprises obtaining a peripheral blood cell (PBMC) sample, culturing the PBMC sample in the presence and absence of a cytokine cocktail including IL-15, L-18, IFNγ, and vitamin D3, contacting said cultured PBMC sample with antibodies that are immunospecific for CD33 expression and intracellular nitric oxide synthase (iNOS), thereby forming detectable immunocomplexes, and comparing detectable iNOS and CD33 expression in cells cultured in the presence and absence of said cytokine cocktail, elevation of CD33⁺, iNOS⁺ cells following treatment being indicative of an IgE⁺ subject prone to systemic inflammation. The method can optionally comprise comparison iNOS levels following cytokine cocktail treatment in PBMC obtained from healthy control IgE⁻ subjects.

In certain embodiments, the cells are monocytes which also express one or more of CD45 and CD14 and elevation of the number of CD33⁺iNOS⁺ cells is associated with IgE positivity and correlate with increased innate immune response activation.

The aforementioned method can be used to advantage in subject in need of monitoring for the development or progression of allergy, asthma, obesity, hypertension, atherosclerosis, coronary artery disease, or stroke.

In yet another aspect, kits are provided which comprises the necessary materials and reagents for practicing the methods described above.

### BRIEF DESCRIPTION OF THE DRAWINGS

FIG. 1. Peripheral blood (PBMC) from adults with asthma rhinoconjunctivitis (n=12, male: 6, female: 6) were cultured for 18 hrs, then expression of CD33⁺iNOS⁺ cells determined (flow cytometry) and its association with current asthma control score (ACT) (Spearman Correlation). There is a significant inverse correlation (R=−0.713, p=0.0093), suggesting that increased expression of CD33⁺iNOS⁺ by peripheral blood leukocytes is associated with more uncontrolled asthma.

FIG. 2. Peripheral blood (PBMC) from adults with asthma rhino conjunctivitis (n=12, male: 6, female: 6) were cultured for 18 hrs, then expression of CD33⁺iNOS⁺ cells determined (flow cytometry) and its association with current asthma quality of life score (AQLQ) (Spearman Correlation). There is a significant inverse correlation (R=−0.667, p=0.0179), suggesting that increased expression of CD33⁺iNOS⁺ by peripheral blood leukocytes is associated with worse quality of life due to asthma.

FIG. 3. Peripheral blood (PBMC) from adults with asthma rhinoconjunctivitis (n=12, male: 6, female: 6) were cultured for 18 hrs, then expression of CD33⁺iNOS⁺ cells determined (flow cytometry) and its association with current rhinoconjunctivitis quality of life score (RQLQ) (Spearman Correlation). There is a significant positive correlation (R=0.651, p=0.0217), suggesting that increased expression of CD33⁺iNOS⁺ by peripheral blood leukocytes is associated with worse quality of life due to rhinoconjunctivitis.

FIG. 4. NIH Asthma Severity Classification of Adult Asthmatics (n=12) correlates with expression of CD33+iNOS+ Cells (% PBMC) (flow cytometry). NIH Asthma Severity—1: Intermittent, 2: Mild Persistent, 3: Moderate Persistent, 4: Severe Persistent, NIH EPR-3) (p=0.014, Pearson Correl.) The data suggests that CD33+iNOS+ percent greater than 10% correlates with persistent asthma which is treated with daily controller medications, including inhaled corticosteroids therapy.

### DETAILED DESCRIPTION OF THE INVENTION

Measurement of nitric oxide responses were initiated after epidemiologic evidence indicated angioedema, is seasonal, with increases most often observed during the summer months. As angioedema is mediated by nitric oxide generation, we began investigating the factors involved in increasing nitric oxide in vitro.

We have found that obesity, as measured by BMI, significantly correlates with both flow cytometry measurements of inducible nitric oxide synthase (iNOS⁺) on CD33⁺ peripheral blood monocytes at 18 hrs, as well as nitric oxide produced by these cells at 5 days for both nonallergic (lgE⁻) and allergic/asthmatic adults (lgE⁺) adults. Attempts to determine NO (as NO₂+NO₃) in serum are possible but extremely unreliable because of diet intake of nitrates. While study subjects are often placed on NO₃ restricted diets, this is difficult to control for.

As there is a significant association of iNOS⁺CD33⁺ levels with nitric oxide produced in culture, this flow cytometric assay can be utilized to measure innate immune response to obesity in associated conditions mediated by nitric oxide, including, but not limited to: allergy/asthma, hypertension, and atherosclerosis.

### Definitions

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an isolated, or biologically pure molecule is a compound that has been removed from its natural milieu.

As used herein “asthma” refers to a respiratory condition marked by spasms in the bronchi of the lungs, causing difficulty in breathing. It usually results from an allergic reaction or other forms of hypersensitivity.

Administration of pharmaceutical preparations for the treatment of inflammatory disorders as asthma, can be in an “effective amount,” this being an amount sufficient to show benefit to the individual. This amount prevents, alleviates, abates, or otherwise reduces the severity of symptoms of anti-inflammatory disorder or condition in a patient.

Agents useful for the treatment of asthma include, without limitation, Albuterol (ProAir HFA, Proventil HFA, Ventolin HFA), Levalbuterol (Xopenex HFA), Metaproterenol, Terbutaline, Prednisone, Prednisolone, and Methylprednisolone. Agents useful for the treatment of rhinoconjunctivitis include antihistamines such as topical levocabastine.

As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15, greater than 20 nucleotides long or greater than 30 nucleotides long.

“Sample” or “patient sample” or “biological sample” generally refers to a sample which may be tested for a particular molecule or cell, preferably a CD33, iNOS⁺ monocyte. Samples may include but are not limited to cells, body fluids, including blood or PBMC preparations, serum, plasma, urine, saliva, tears, pleural fluid and the like.

### Kits for Practicing the Methods of the Invention

Kits are also provided to facilitate the practice of the methods described herein. In one embodiment, the kit comprises antibodies immunologically specific for CD33 and iNOS, with or without non-naturally occurring detectable labels immobilized thereon; one or more cytokines, alone or in combination selected from vitamin D3, IL-15, IL-18, IFNγ. Each of these reagents is commercially available from vendors which include, without limitation, Sigma Aldrich, Abcam, BD Sciences and Invitrogen. Suitable labels include, for example, PE, AlexaFluor, and GFP, YFP, BFP, and CFP comprising non-naturally occurring mutations which enhance fluorescence or stability of the fluorophore.

Kits of the invention may also comprise reagents suitable for PBMC preparation (e.g, Ficoll-Hypaque disposable pipette, culture plates, culture medium suitable for culture of the isolated (e.g., RMPI), treated cells, reagents for flow cytometry, sterile PBS,

The following examples are provided to illustrate certain embodiments of the invention. They are not intended to limit the invention in any way.

### Example I

### Cytokines (IL-15, IL-18, IFNγ) and Vitamin D3 Increase Expression of Inducible Nitric Oxide Synthase (iNOS) by Monocytes from IgE⁺ Adults with Asthma

Data from earlier experiments revealed that PBMC from IgE⁺ (>100 IU/ml) adults with asthma produced significantly greater nitric oxide (NO) concentrations than IgE⁻ (<100 IU/ml) after 5 days in culture. NO production further increased in the presence of cytokines (IL-15, IL-18, IFNγ) and vitamin D3 (CYT+D3). We examined intracellular iNOS expression in CD33⁺ monocytes from IgE⁺ and IgE⁻ adults after 18 hrs CYT+D3.

Blood from IgE⁺ (n=14) and IgE⁻ (n=7) adults was collected. Exhaled breath NO and serum IgE levels were determined (Niox Vero; fluoroenzyme immunoassay). PBMC were incubated for 18 hrs±hIL-15 (1 μg/ml), IL-18 (1 μg/ml), IFNγ (10 ng/ml) and vitamin D3 (20 pmol/ml). Cells were collected by vigorous pipetting and analyzed by flow cytometry (Fortessa) for surface CD33 expression (anti-CD33 PE, BD Biosciences) and intracellular iNOS expression (rabbit monoclonal anti-human iNOS) (abcam), followed by goat anti-rabbit IgG (Alexa Fluor 488) (abcam). Spearman and Pearson coefficients, t-test, and Mann-Whitney U-test were used in analysis.

Monocytes in peripheral blood were CD45⁺CD14⁺ (92.6, ±3.4%) and CD33⁺ (96.5, ±3.7%), and iNOS⁻ (<2%) (n=5). Incubation for 18 hrs with CYT+D3 significantly increased CD33⁺iNOS⁺ monocytes in PBMC from IgE+ adults (27.5 vs 15.65%; p<0.016), but not iNOS expression in PBMC from IgE⁻ subjects (6.5 vs 8.7%; p>0.05) (Mann-Whitney U-test)

Measurement of intracellular iNOS by flow cytometry in CD33⁺ monocytes reveals increased expression in the presence of cytokines and vitamin D3 in IgE⁺ adults. Accordingly, monocyte iNOS expression can be used to advantage as a biomarker of systemic inflammation in IgE⁺ adults with asthma.

### Example II

The following methods were used to generate the data described in Example II.

Adults with asthma, with or without rhinoconjunctivitis symptoms (n=12) completed asthma control and QOL questionnaires (ACT, AQLQ, RQLQ) and had AM blood drawn for peripheral blood measurement of CD33 and iNOS (flow cytometry). PBMC were incubated for 18 hrs and then cells were collected by vigorous pipetting and analyzed for surface CD33 expression (anti-CD33 PE, BD Biosciences) and intracellular iNOS expression (rabbit monoclonal anti-human iNOS) (abcam), followed by goat anti-rabbit IgG (Alexa Fluor 488) (abcam) using flow cytometry (LSR Fortessa). Spearman and Pearson coefficients were generated.

### Demographic of Study Subjects

6 men, 6 women

Average age (yrs): 47.8±15.0

Total serum IgE (nl<100 IU/ml): 499.1±492.6

Asthma severity*:
Intermittent: 5
Mild persistent: 2
Moderate persistent: 4
Severe Persistent: 1

*NIH EPR-3 Guidelines, 2007

Treatment: All 5 patients with Intermittent asthma are managed with Beta-agonist inhaler use as needed.

The remaining 7 patients with persistent asthma require controller therapy including inhaled corticosteroids.

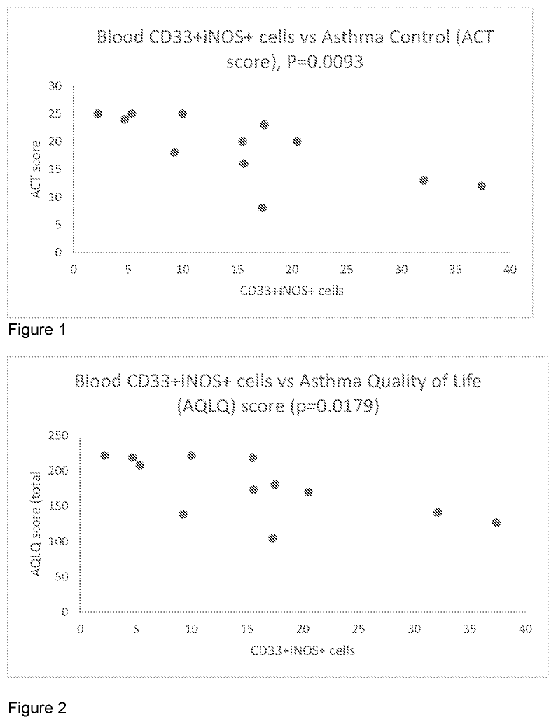

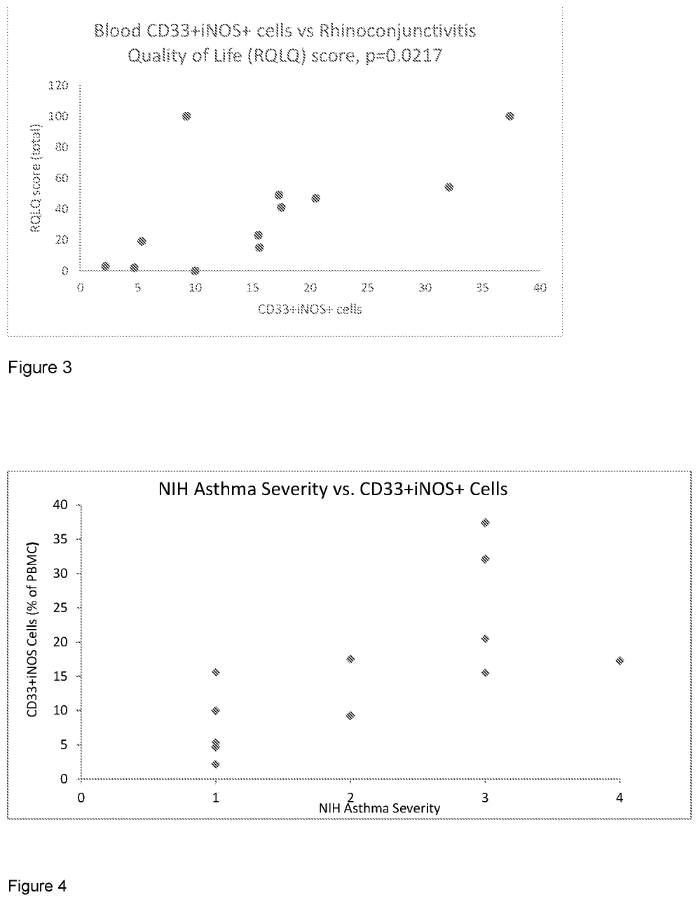

We have determined that peripheral blood measurements of CD33⁺iNOS⁺ cells by flow cytometry significantly correlate with asthma control, as measured by the Asthma Control Test (a validated, short patient survey, recommended in the EPR-3 Asthma Guidelines (NIH)) to help in adjustment of asthma therapy. Higher levels of CD33+iNOS+ cells are associated with lower asthma control as shown in FIGS. 1-4. ACT scores of <19 indicate poorly controlled asthma, and correlate with approximate measurement of at least 5%-10%, preferably greater than 20% CD33+iNOS+ cells in the PBMC preparation. According to the NIH, this would be an indication for change in stepwise therapy. Further, CD33⁺iNOS⁺ measurements also significantly correlate with validated QOL scores for both asthma (Asthma Quality of Life (AQLQ)) and rhinoconjunctivitis (Rhinoconjunctivitis Quality of Life (RQLQ)), with lower AQLQ scores indicating poor QOL from asthma correlating with higher CD33⁺ iNOS⁺ measurement, and higher RQLQ indicating worse QOL from rhinoconjunctivitis similarly associated with increased measurements of CD33⁺iNOS⁺. All three clinical scores are in line with higher levels of CD33⁺iNOS⁺ measurements correlating with worse asthma control and poorer QOL associated with asthma and rhinoconjunctivitis.

These data indicate that measurement of blood CD33⁺iNOS⁺ cells (flow cytometry) can be used to advantage in guiding treatment as a biomarker of asthma and rhino-conjunctivitis.

### Example III

### Adult Body Mass Index (BMI) Correlates with Both CD33+ Monocyte iNOS Responses and Nitric Oxide Levels In Vitro

As mentioned above, obesity, as measured by BMI, is associated with asthma severity and adipocyte macrophages iNOS responses. While BMI does not correlate with FeNO, a biomarker of allergic asthma, it is not known how BMI relates to blood monocyte nitric oxide responses in adults±allergy/asthma.

BMI, AM FeNO, and total serum IgE levels determined (Niox Vero; lmmunoCAP fluoroenzymeimmunoassay, respectively) were determined for adults (allergy/asthma n=13, BMI 30.6±5.8, FeNO 45.8 ppb±40.7, IgE 462.1 IU/ml±472.1; control n=11, BMI 25.9±2.1, FeNO14.6 ppb 7.9). PBMC were isolated from venous blood by density gradient centrifugation and incubated for 18 hrs (±hIL-15 (1 μg/ml), ±IL-18 (1 μg/ml), IFNγ (10 ng/ml) and vitamin D3 (20 pmol/ml) (cyto/vit D). Cells were collected by vigorous pipetting and analyzed for surface CD33 expression (anti-CD33 PE, BD Biosciences) and intracellular iNOS expression (rabbit monoclonal anti-human iNOS) (Abeam), followed by goat anti-rabbit lgG (Alexa Fluor 488) (abcam) using flow cytometry (LSR Fortessa). NO production was measured using Griess reaction on day 5. Spearman coefficients were used in analysis.

The results show that while BMI does not correlate with FeNO (p=0.54), BMI significantly correlates with expression of iNOS on cyto/vit D stimulated CD33⁺ cells (p=0.008), but not unstimulated CD33⁺ cells (p=0.06). Both BMI and expression of iNOS at 18 hrs from cyto/vit D stimulated CD33⁺ cell significantly correlates with NO produced at day 5 (p=0.0017, 0.002, respectively).

These data indicate that the inflammatory responses associated with obesity also involve blood nitric oxide responses, including those of adults with allergy/asthma.

### REFERENCES

National Heart, Lung, and Blood Institute National Asthma Education and Prevention Program Expert Panel Report 3: Guidelines for the Diagnosis and Management of Asthma. August 2007.
Nathan R A. Development of the Asthma Control Test J Allergy Clin Immunol 2004. 113 (1): 59-65.
Juniper E F, Guyatt G H, Epstein R S, Ferrie P J, Jaeschke R, Hiller T K. Evaluation of Impairment of Health Related Quality-of-Life in Asthma—Development of a Questionnaire for Use in Clinical-Trials. Thorax 1992; 47(2):76-83.
Juniper E F, Guyatt G H. Development and testing of a new measure of health status for clinical trials in rhinoconjunctivitis. Clin Experimental Allergy 1991; 21:77-83.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for detecting iNOS expression levels in CD33⁺ monocytes as a biomarker for uncontrolled asthma in IgE⁺ subjects, comprising;
a) obtaining a peripheral blood cell (PBMC) sample from said subject,
b) contacting said PBMC sample with antibodies that are immunospecific for CD33 and intracellular nitric oxide synthase (iNOS), thereby forming detectable immunocomplexes,
d) measuring the percentage of monocytes in the sample which are CD33⁺ and iNOS⁺, the presence of 10% or greater of CD33⁺iNOS⁺ monocytes being indicative of poor asthma control and the need for additional therapy to manage uncontrolled asthma symptoms.

2. The method of claim 1, wherein CD33 and iNOS expression is measured in monocytes that express one or more of CD45 and CD14.

3. The method of claim 1, wherein the antibodies specific for iNOS are fluorescently tagged with non-naturally occurring detectable labels and detected by flow cytometry or ELISA.

4. The method of claim 1, wherein the levels of iNOS are associated with IgE positivity and increased innate immune response activation.

5. A method for detecting iNOS expression levels in CD33⁺ monocytes as a biomarker for systemic inflammation in IgE⁺ subjects, comprising;
a) obtaining a peripheral blood cell (PBMC) sample from said subject,
b) culturing said PBMC sample in the presence and absence of a cytokine cocktail including IL-15, IL-18, IFNγ, and vitamin D3;
c) contacting said cultured PBMC sample with antibodies that are immunospecific for CD33 and intracellular nitric oxide synthase (iNOS), thereby forming detectable immunocomplexes,
d) comparing detectable iNOS and CD33 immunocomplex levels cultured in the presence and absence of said cytokine cocktail, elevation of CD33+, iNOS+ cells in the presence of said cocktail being indicative of an increased risk for systemic inflammation in an IgE+ subject.

6. The method of claim 5, wherein the subject is being monitored for the development or progression of allergy, asthma, rhino-conjunctivitis, obesity, hypertension, atherosclerosis, coronary artery disease, or stroke.

7. The method of claim 5, further comprising comparison of cytokine cocktail treatment in PBMC obtained from healthy control IgE− subjects.

8. A kit comprising materials and compositions for practicing the method of claim 1.

9. A kit comprising materials and compositions for practicing the method of claim 5.
